(19) United States
(12) Patent Application Publication
(10) Pub. No.: US 2018/0016304 A1
(43) Pub. Date: Jan. 18, 2018

(54) SELF-ASSEMBLING ULTRASHORT ALIPHATIC DEPSIPEPTIDES FOR BIOMEDICAL APPLICATIONS

(71) Applicant: Agency for Science, Technology and Research, Singapore (SG)

(72) Inventors: Charlotte A. E. Hauser, Singapore (SG); Michael R. Reithofer, Singapore (SG); Yihua Loo, Singapore (SG)

(73) Assignee: Agency for Science, Technology and Research, Singapore (SG)

(21) Appl. No.: 15/549,874

(22) PCT Filed: Mar. 3, 2016
(86) PCT No.: PCT/SG2016/050100

§ 371 Date: Aug. 9, 2017

(30) Foreign Application Priority Data
Mar. 6, 2015 (SG) 10201501744S

(51) Int. Cl. C07K 11/00; A61K 47/34; A61L 27/52; A61L 27/54; A61L 27/22

(52) U.S. Cl. CPC C07K 11/00; A61L 27/54; A61L 27/227; A61L 27/52; A61K 47/34; A61L 2400/06; A61L 2430/34; G01N 2800/7095

(57) ABSTRACT

The present invention relates to ultrashort depsipeptides which are capable of self-assembling into hydrogels. One preferred embodiment is of Ac-ILVaGK-NH₂, where a represents lactic acid. The invention also relates to the use of these depsipeptides in formulating hydrogels, co-gels or co-hydrogels, and pharmaceutical compositions or biomedical device or surgical implants or kits comprising these depsipeptides for various therapeutic applications such as regenerative medicine, tissue regeneration and tissue re-placement.

### FIELD OF INVENTION

The present invention is in the area of cell and tissue engineering and nanomedicine. The invention generally relates to depsipeptides and their use in hydrogels as well as in co-gels or co-hydrogels.

### BACKGROUND

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

The development of easily biodegradable hydrogel-forming peptides has been a long term desire. Early studies by Zhang et al. (1993) have reported that β-sheet forming peptides such as (AEAEAKAK)₂ contain amino acid sequences expected to be enzymatically digested but instead remained undigested when exposed to α-chymotrypsin, trypsin, and papain. In an attempt to overcome these drawbacks several peptides which bare a peptide sequence that can be cleaved by matrix metalloproteinases (MMPs) have been reported (Chau et al., 2008; Galler et al., 2010; Kumada et al., 2010; Giano et al., 2011; Jun et al., 2005). Although this strategy has yielded biodegradable β-sheet fibrillizing peptides, it is difficult to predict and control the amount of in vivo degradation over time due to varying MMP concentrations.

Hydrolytically susceptible materials have drawn significant attention over the last decades, taking their predictable enzyme independent degradation into consideration (Freed et al., 1994; Ishaug et al., 1997; Kim and Mooney, 1998; Lynn et al., 2001; Ron et al., 1993).

Although ester hydrolysis is commonly used in biopolymers, only two recent reports show a similar approach for peptides: Tian et al. (2013) report the synthesis and degradation abilities of four depsipeptides comprising 11 amino acids. In this study, the peptide sequences utilized contained both aliphatic and aromatic amino acids and are zwitterionic in nature. The different α-hydroxy acids had an impact on the degradation time. In a second report, Nguyen et al. (2014) described the synthesis and self-assembling properties of Fmoc-protected depsipeptides. Here, charged residues K and D were alternated with hydrophobic lactic acid, in a fashion similar to peptides that form β-sheets. Although self-assembly was observed, the authors attributed it to the π-π stacking of the Fmoc group, and thus only Fmoc protected depsipeptides were investigated.

There is a need in the art of cell and tissue engineering and nanomedicine for improved means and methods.

### SUMMARY

The present technology proposes ultrashort aliphatic depsipeptides which are capable of self-assembling into hydrogels. The present invention comprises the following features:

F1) Ultrashort aliphatic depsipeptide (such as Ac-ILVaGK-NH2; a=lactic acid; SEQ ID NO. 52) with the following features:
Presence of at least one ester linkage;
Increasing biodegradation via hydrolysis;
Peptides sequence (such as LIVAGK, ILVAGK, LIVAGD, AIVAGS, IVDA, IVAD, IVA, ILA, IVKA), arranged in order of decreasing hydrophobicity;
Self-assembly;
Stimuli responsive (i.e. salt concentration; pH, ionic concentration, depsipeptide concentration);
Non-toxic;
Bioactive alphα-hydroxy acid moiety can be selected to exert biological effects on cells/ tissues (i.e. exfoliation; anti-aging); and
Can be mixed with the ‘parent’ ultrashort peptides to create co-gels (well-controlled bulk stability and biodegradation).

F2) The peptide as of F1 that has applications in:
Drug/gene delivery, injectable therapies, bio-printing, cell encapsulation, cosmetics, and other biological uses.

This disclosure describes a technology to synthesize ultrashort aliphatic depsipeptides which are capable of self-assembling into hydrogels in aqueous conditions. The synthesized depsipeptides demonstrate stimuli responsive properties—increasing salt concentration significantly reduce the minimum gelation concentration. Furthermore, cytocompatibility studies show that the material is nontoxic. These depsipeptides undergo hydrolysis, yielding smaller fragments that do not support self-assembly. The ability of the gel to “dissolve” can be exploited for biomedical applications where degradation of the hydrogel scaffold is needed. The bioactive α-hydroxy acid moiety can be carefully selected to exert biological/biochemical effects on cells/tissues (e.g. exfoliation). The depsipeptide can also be mixed with the parent ultrashort peptide to create co-gels, whose bulk stability and biodegradation rate can be fairly well-controlled by the relative composition of both constituents.

The presence of ester linkage (versus amide bond) increases the in vitro and in vivo biodegradation via hydrolysis in a biological setting. The α-hydroxy acids present in the structure can exert biological effects (such as exfoliation and anti-ageing properties) leading to new biomedical applications. The peptidic portion gives rise to scaffold structures due to self-assembly, and thus can provide sustained delivery of the bioactive portion. The resulting hydrogel can provide sustained and controlled release of the bioactive α-hydroxy acid. The depsipeptide can be mixed with the parent ultrashort peptide to create co-gels, whose bulk stability and biodegradation rate can be controlled by relative composition of constituents.

In accordance with an aspect of the present invention, the invention provides a depsipeptide, capable of self-assembling and forming a hydrogel, having the general formula selected from general formula I, II and III:

Zₐ-(X^{AHA})_{b}-(Y)_{c}-Z′_{d} (I)
wherein
Z is an N-terminal protecting group;
a is at least 1,
X^{AHA} is, at each occurrence, independently selected from the group consisting of aliphatic amino acids and aliphatic amino acid derivatives and includes at least one α-hydroxy acid;
   b is an integer selected from 2 to 7;
Y is selected from the group consisting of polar amino acids and polar amino acid derivatives;
   c is 0, 1 or 2;
Z′ is a C-terminal protecting group; and
d is 0 or 1, and
b+c is at least 3;

Zₐ-(X)_{b1}-(AHA)_{d}-(X)_{b2}-(Y)_{c}-Z′ₑ (II)
wherein
Z is an N-terminal protecting group;
   a is at least 1;
X is, at each occurrence, independently selected from the group consisting of aliphatic amino acids and aliphatic amino acid derivatives;
   with each of b1 and b2 being an integer selected from 0 to 7, under the proviso that b1+b2 is 2 to 7,
AHA is, at each occurrence, independently selected from the group consisting of α-hydroxy acids;
   d is 1 or 2;
Y is selected from the group consisting of polar amino acids and polar amino acid derivatives;
   c is 0, 1 or 2;
   under the proviso that b1+b2 +c +d <7,
Z′ is a C-terminal protecting group; and
e is 0 or 1;

Zₐ-(X)_{b′}-(Y)_{c}-(AHA)_{d}-Z′ₑ (III)
wherein
Z is an N-terminal protecting group;
a is at least 1;
X is, at each occurrence, independently selected from the group consisting of aliphatic amino acids and aliphatic amino acid derivatives;
   b′ is an integer selected from 2 to 7;
Y is selected from the group consisting of polar amino acids and polar amino acid derivatives;
   c is 0, 1 or 2;
AHA is, at each occurrence, independently selected from the group consisting of α-hydroxy acids;
   d is 1 or 2;
Z′ is a C-terminal protecting group; and
e is 0 or 1, and
b′ +c is at least 2.

In one embodiment, the α-hydroxy acid is selected from lactic acid, glycolic acid, malic acid, 2,3-dihydroxypropanoic acid, lactobionic acid, and citric acid.

In one embodiment, said aliphatic amino acids and aliphatic amino acid derivatives, and said polar amino acids and polar amino acid derivatives are either D-amino acids or L-amino acids,

and/or said α-hydroxy acids are corresponding to their natural amino acids either in L or in D form.

In one embodiment, said aliphatic amino acids are selected from the group consisting of alanine (Ala, A), homoallylglycine, homopropargylglycine, isoleucine (Ile, I), norleucine, leucine (Leu, L), valine (Val, V) and glycine (Gly, G), preferably from the group consisting of alanine (Ala, A), isoleucine (Ile, I), leucine (Leu, L), valine (Val, V) and glycine (Gly, G).

In one embodiment, all or a portion of said aliphatic amino acids are arranged in an order of decreasing amino acid size in the direction from N- to C-terminus, wherein the size of the aliphatic amino acids is defined as I=L>V>A>G,

and/or wherein the overall hydrophobicity decreases from N- to C-terminus.

In one embodiment, (X^{AHA})_{b} of formula I or (X)_{b1}-(AHA)_{d}-(X)_{b2} of formula II has a sequence selected from

(SEQ ID NO: 1)
LIVAG(AHA),
(SEQ ID NO: 2)
LIVA(AHA),
(SEQ ID NO: 3)
LIV(AHA)G,
(SEQ ID NO: 4)
ILVAG(AHA),
(SEQ ID NO: 5)
ILVA(AHA),
(SEQ ID NO: 6)
ILV(AHA)G,
(SEQ ID NO: 7)
IVLA(AHA),
(SEQ ID NO: 8)
IVL(AHA)G,
(SEQ ID NO: 9)
AIVAG(AHA),
(SEQ ID NO: 10)
AIVA(AHA),
(SEQ ID NO: 11)
AIV(AHA)G,
IV(AHA),
IL(AHA),

wherein, optionally, there is an A preceding such sequence at the N-terminus, and wherein AHA refers to α-hydroxy acid.

Preferably, (X^{AHA})_{b} of formula I or (X)_{b1}-(AHA)_{d}-(X)_{b2} of formula II has a sequence selected from

(SEQ ID NO: 12)
LIVAg,
(SEQ ID NO: 13)
LIVaG,
(SEQ ID NO: 14)
LIVAm,
(SEQ ID NO: 15)
LIVAGm,
(SEQ ID NO: 16)
ILVAg,
(SEQ ID NO: 17)
ILVaG,
(SEQ ID NO: 18)
ILVAm,
(SEQ ID NO: 19)
ILVAGm,
(SEQ ID NO: 20)
IVLAg,
(SEQ ID NO: 21)
IVLaG,
(SEQ ID NO: 22)
AIVAg,
(SEQ ID NO: 23)
AIVaG,
(SEQ ID NO: 24)
AIVAGm,
IVa,
ILa,
IVm,

wherein “g” refers to glycolic acid, “a” refers to lactic acid and “m” refers to malic acid, wherein, optionally, there is an A preceding such sequence at the N-terminus.

In one embodiment, (X)_{b′} of formula III has a sequence selected from IV, wherein, optionally, there is an A preceding such sequence at the N-terminus,

or (X)_{b′} of formula III has a sequence selected from

(SEQ ID NO: 25)
LIVAG,
(SEQ ID NO: 26)
ILVAG,
(SEQ ID NO: 27)
LIVAA,
(SEQ ID NO: 28)
LAVAG,
(SEQ ID NO: 29)
AIVAG
(SEQ ID NO: 30)
GIVAG
(SEQ ID NO: 31)
VIVAG
(SEQ ID NO: 32)
ALVAG
(SEQ ID NO: 33)
GLVAG
(SEQ ID NO: 34)
VLVAG
(SEQ ID NO: 35)
IVAG
(SEQ ID NO: 36)
LIVA
(SEQ ID NO: 37)
LIVG
and
IVA,

wherein, optionally, there is an A preceding such sequence at the N-terminus.

In one embodiment,
in formula I b is an integer from 2 to 7, preferably 3 to 7 or 3 to 6 or 2 to 6, or more preferably 2 to 5,
in formula II b1+b2 is 2 to 7, preferably 3 to 7 or 3 to 6 or 2 to 6, or more preferably 2 to 5, and
in formula III b′ is an integer from 2 to 7, preferably 3 to 7 or 3 to 6 or 2 to 6, or more preferably 2 to 5.

In one embodiment, said polar amino acids are selected from the group consisting of aspartic acid (Asp, D), asparagine (Asn, N), glutamic acid (Glu, E), glutamine (Gln, Q), 5-N-ethyl-glutamine (theanine), citrulline, thio-citrulline, cysteine (Cys, C), homocysteine, methionine (Met, M), ethionine, selenomethionine, telluromethionine, threonine (Thr, T), allothreonine, serine (Ser, S), homoserine, arginine (Arg, R), homoarginine, ornithine (Orn), lysine (Lys, K), N(6)-carboxymethyllysine, histidine (His, H), 2,4-diaminobutyric acid (Dab), 2,3-diaminopropionic acid (Dap), and N(6)-carboxymethyllysine,

wherein said polar amino acid is preferably selected from the group consisting of aspartic acid, asparagine, glutamic acid, glutamine, serine, threonine, methionine, lysine, ornithine (Orn), 2,4-diaminobutyric acid (Dab), and 2,3-diaminopropionic acid (Dap).

In one embodiment,

c is 2 and said polar amino acids are identical amino acids,

or c is 1 and said polar polar amino acid comprises any one of aspartic acid, asparagine, glutamic acid, glutamine, serine, threonine, cysteine, methionine, lysine, ornithine, 2,4-diaminobutyric acid (Dab) and histidine,

preferably lysine, ornithine, 2,4-diaminobutyric acid (Dab) and 2,3-diaminopropionic acid (Dap).

In one embodiment, (Y)_{c} has a sequence selected from Asp, Asn, Glu, Gln, Ser, Thr, Cys, Met, Lys, Orn, Dab, His, Asn-Asn, Asp-Asp, Glu-Glu, Gln-Gln, Asn-Gln, Gln-Asn, Asp-Gln, Gln-Asp, Asn-Glu, Glu-Asn, Asp-Glu, Glu-Asp, Gln-Glu, Glu-Gln, Asp-Asn, Asn-Asp Thr-Thr, Ser-Ser, Thr-Ser, Ser-Thr, Asp-Ser, Ser-Asp, Ser-Asn, Asn-Ser, Gln-Ser, Ser-Gln, Glu-Ser, Ser-Glu, Asp-Thr, Thr-Asp, Thr-Asn, Asn-Thr, Gln-Thr, Thr-Gln, Glu-Thr, Thr-Glu, Cys-Asp, Cys-Lys, Cys-Ser, Cys-Thr, Cys-Orn, Cys-Dab, Cys-Dap, Lys-Lys, Lys-Ser, Lys-Thr, Lys-Orn, Lys-Dab, Lys-Dap, Ser-Lys, Ser-Orn, Ser-Dab, Ser-Dap, Orn-Lys, Orn-Orn, Orn-Ser, Orn-Thr, Orn-Dab, Orn-Dap, Dab-Lys, Dab-Ser, Dab-Thr, Dab-Orn, Dab-Dab, Dab-Dap, Dap-Lys, Dap-Ser, Dap-Thr, Dap-Orn, Dap-Dab, Dap-Dap.

In one embodiment, (X^{AHA})_{b}-(Y)_{c} of formula I or (X)_{b1}-(AHA)_{d}-(X)_{b2}-(Y)_{c} of formula II has a sequence selected from the group consisting of

(SEQ ID NO: 38)
LIVA(AHA)K,
(SEQ ID NO: 39)
LIV(AHA)GK,
(SEQ ID NO: 40)
LIVA(AHA)D,
(SEQ ID NO: 41)
LIV(AHA)GD,
(SEQ ID NO: 2)
LIVA(AHA),
(SEQ ID NO: 1)
LIVAG(AHA),
(SEQ ID NO: 42)
ILVA(AHA)K,
(SEQ ID NO: 43)
ILV(AHA)GK,
(SEQ ID NO: 5)
ILVA(AHA),
(SEQ ID NO: 4)
ILVAG(AHA),
(SEQ ID NO: 44)
IVLA(AHA)K,
(SEQ ID NO: 45)
IVL(AHA)GK,
(SEQ ID NO: 46)
AIVA(AHA)S,
(SEQ ID NO: 47)
AIV(AHA)GS,
(SEQ ID NO: 9)
AIVAG(AHA),
(SEQ ID NO: 48)
IV(AHA)D,
IV(AHA),
and
IL(AHA).

Preferably, (X^{AHA})_{b}-(Y)_{c} of formula I or (X)_{b1}(AHA)_{d}-(X)_{b2}-(Y)_{c} of formula II has a sequence selected from the group consisting of

(SEQ ID NO: 49)
LIVAgK,
(SEQ ID NO: 50)
LIVaGK,
(SEQ ID NO: 51)
ILVAgK,
(SEQ ID NO: 52)
ILVaGK,
(SEQ ID NO: 53)
IVLAgK,
(SEQ ID NO: 54)
IVLaGK,
(SEQ ID NO: 55)
LIVAgD,
(SEQ ID NO: 56)
LIVaGD,
(SEQ ID NO: 57)
AIVAgS
(SEQ ID NO: 58)
AIVaGS
(SEQ ID NO: 59)
IVaD
(SEQ ID NO: 14)
LIVAm,
(SEQ ID NO: 18)
ILVAm,
(SEQ ID NO: 24)
AIVAGm,
IVa,
IVm,
and
ILa.

In one embodiment, (X)_{b′}-(Y)_{c}-(AHA)_{d} of formula III has a sequence selected from the group consisting of

(SEQ ID NO: 60)
IVD(AHA),
(SEQ ID NO: 61)
IVK(AHA),
(SEQ ID NO: 62)
ILVAGK(AHA),
(SEQ ID NO: 63)
ILVAGD(AHA),
(SEQ ID NO: 64)
ILVAGS(AHA),
(SEQ ID NO: 65)
LIVAGK(AHA),
(SEQ ID NO: 66)
LIVAGD(AHA),
(SEQ ID NO: 67)
LIVAGS(AHA),
and
(SEQ ID NO: 68)
AIVAGS(AHA).

Preferably, (X)_{b′}-(Y)_{c}-(AHA)_{d} of formula III has a sequence selected from the group consisting of

(SEQ ID NO: 69)
IVDa,
(SEQ ID NO: 70)
IVKa.

In one embodiment, a is 1 and said N-terminal protecting group Z has the general formula —C(O)—R, wherein R is selected from the group consisting of H, unsubstituted or substituted alkyls, and unsubstituted or substituted aryls,.

wherein R is preferably selected from the group consisting of methyl, ethyl, propyl, isopropyl, butyl and isobutyl.

In one embodiment, said N-terminal protecting group Z is an acetyl group.

In one embodiment, said N-terminal protecting group Z is a peptidomimetic molecule, including natural and synthetic amino acid derivatives, wherein the N-terminus of said peptidomimetic molecule may be modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

In one embodiment, said C-terminal protecting group Z′ is an amide group or an ester group.

Preferably, said C-terminal protecting group Z′ is an amide group and the C-terminus of the depsipeptide has the formula —CONHR or —CONRR′, with R and R′ being selected from the group consisting of H, unsubstituted or substituted alkyls, and unsubstituted or substituted aryls.

Preferably, said C-terminal protecting group Z′ is an ester group and the C-terminus of the depsipeptide has the formula —CO₂R , with R being selected from the group consisting of H, unsubstituted or substituted alkyls, and unsubstituted or substituted aryls.

In one embodiment, said C-terminal protecting group Z′ is a peptidomimetic molecule, including natural and synthetic amino acid derivatives, wherein the C-terminus of said peptidomimetic molecule may be modified with a functional group selected from the group consisting of carboxylic acid, amide, alcohol, aldehyde, amine, imine, nitrile, an urea analog, thiol, phosphate, carbonate, sulfate, nitrate, maleimide, vinyl sulfone, azide, alkyne, alkene, carbohydrate, imide, peroxide, ester, thioester, aryl, ketone, sulphite, nitrite, phosphonate and silane.

In accordance with an aspect of the present invention, the invention provides a method of preparing a hydrogel, the method comprising dissolving at least one depsipeptide as defined in any one of claims 1 to 23 in an aqueous solution.

In one embodiment, the method comprises stimuli-responsive gelation of the at least one depsipeptide as defined herein,

wherein said stimulus/stimuli or gelation condition(s) is/are selected from salt concentration, pH, ionic concentration and/or depsipeptide concentration.

Preferably, gelation is carried out in the presence of salt at physiological conditions (such as PBS, or 0.9% saline and PBS).

In one embodiment, the at least one depsipeptide is dissolved at a concentration from 10 mg/mL to 500 mg/mL, preferably at a concentration from 50 mg/mL to 150 mg/mL, more preferably at a concentration of about 60 mg/mL or about 100 mg/mL.

In one embodiment, the method comprises the addition of further compound(s) prior or during gelation/self-assembly, which are encapsulated by the hydrogel,
wherein said further compound(s) can be selected from
   bioactive molecules or moieties,
      such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
   label(s), dye(s),
      such as imaging contrast agents;
   pathogens,
      such as viruses, bacteria and parasites;
   quantum dots, nano- and microparticles,
   or combinations thereof.

In one embodiment, the method comprises the addition or mixing of cells prior or during gelation/self-assembly, which are encapsulated by the hydrogel,
wherein said cells can be stem cells (mesenchymal, progenitor, embryonic and induced pluripotent stem cells), transdifferentiated progenitor cells and primary cells isolated from patient samples (fibroblasts, nucleus pulposus).

preferably comprising the addition of further compound(s) prior or during gelation (such as defined above), which are co-encapsulated by the hydrogel,

optionally comprising the addition or mixing of different cells prior or during gelation/self-assembly and/or comprising the addition or mixing of cells onto the hydrogel after gelation.

Preferably, the method comprises the following steps:

(1) the addition or mixing of cells prior or during gelation, which are encapsulated by the hydrogel, and

(2) subsequently the addition of cells onto the printed hydrogel,

wherein said cells of (1) and (2) are the same or different, and can be stem cells (adult, progenitor, embryonic and induced pluripotent stem cells), transdifferentiated progenitor cells, and primary cells (isolated from patients) and cell lines (such as epithelial, neuronal, hematopoietic and cancer cells).

In one embodiment, the method comprises comprising the use of different depsipeptides.

In accordance with an aspect of the present invention, the invention provides a method of preparing a co-gel or co-hydrogel, the method comprising

(a) dissolving at least one depsipeptide of the present invention in an aqueous solution, preferably under the conditions as defined herein above,

(b) dissolving at least one peptide which has the same sequence as the depsipeptide of step (a), but includes no AHA (“parent peptide”), in an aqueous solution,

(c) mixing the solutions of (a) and (b) and gelating, preferably stimuli-responsive gelation as defined herein above,

(d) obtaining the co-gel or co-hydrogel.

In accordance with an aspect of the present invention, the invention provides a hydrogel comprising at least one depsipeptide of the present invention, preferably obtained by the method of the present invention.

In one embodiment, the hydrogel has a lower degradation stability compared to the hydrogel with the parent peptide, i.e. the peptide which has the same sequence as the depsipeptide but includes no AHA.

In one embodiment, the hydrogel is stable in aqueous solution at ambient temperature for a period of at least 7 days, preferably at least 2 to 4 weeks, more preferably at least 1 to 6 months.

In one embodiment, the hydrogel is characterized by a storage modulus G′ to loss modulus G″ ratio that is greater than 2.

In one embodiment, the hydrogel is characterized by a storage modulus G′ from 100 Pa to 80,000 Pa at a frequency in the range of from 0.02 Hz to 16 Hz.

In one embodiment, the hydrogel has tuneable mechanical properties, such as a stiffness which can be tuned by varying pH, ionic concentration and depsipeptide concentration.

In accordance with an aspect of the present invention, the invention provides a co-gel or co-hydrogel comprising

at least one depsipeptide of the present invention, and

at least one parent peptide, i.e. a peptide which has the same sequence as the depsipeptide, but includes no AHA,

preferably obtained by the method of the present invention.

In one embodiment, the co-gel or co-hydrogel of the present invention has a lower degradation stability compared to the hydrogel comprising the parent peptide, i.e. the peptide which has the same sequence as the depsipeptide but includes no AHA, but not the depsipeptide

In one embodiment, the hydrogel of the present invention or the co-gel or co-hydrogel of the present invention furthermore comprise:

further compound(s), which are encapsulated by the hydrogel or the co-gel or co-hydrogel, wherein said further compound(s) can be selected from
bioactive molecules or moieties,
   such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
label(s), dye(s),
   such as imaging contrast agents;
pathogens,
   such as viruses, bacteria and parasites;
quantum dots, nano- and microparticles,
or combinations thereof;

and/or

cells, which are encapsulated by the hydrogel or the co-gel or co-hydrogel and/or added onto the hydrogel or the co-gel or co-hydrogel after gelation

wherein said cells are the same or different, and can be stem cells (adult, progenitor, embryonic and induced pluripotent stern cells), transdifferentiated progenitor cells, and primary cells (isolated from patients) and cell lines (such as epithelial, neuronal, hematopoietic and cancer cells).

In accordance with an aspect of the present invention, the invention provides a pharmaceutical and/or cosmetic composition and/or a biomedical devive and/or a surgical implant comprising

at least one depsipeptide of the present invention,

a hydrogel of the present invention, or

a co-gel or co-hydrogel of the present invention.

In one embodiment, the pharmaceutical and/or cosmetic composition and/or the biomedical device and/or the surgical implant of the present invention further comprise a pharmaceutically active compound, and optionally a pharmaceutically acceptable carrier.

In one embodiment, the pharmaceutical and/or cosmetic composition is injectable.

In accordance with an aspect of the present invention, the invention provides a kit of parts, the kit comprising

a first container with at least one depsipeptide of the present invention, and

a second container with an aqueous solution,
wherein optionally the first and/or second contained further comprises a pharmaceutically active compound,

optionally, a third container with a gelation enhancer,

wherein said gelation enhancer is preferably a salt or a solution of a salt.

In one embodiment, the kit of parts further comprises

a fourth container with at least one parent peptide of the at least one depsipeptide of the first container, and

a fifth container with an aqueous solution.

In one embodiment, at least one of said first, second, third, fourth or fifth container is provided as a spray bottle or a syringe.

In accordance with an aspect of the present invention, the invention provides the use of a depsipeptide of the present invention, a hydrogel of the present invention, a co-gel or co-hydrogel of the present invention, or a pharmaceutical and/or cosmetic composition and/or a biomedical device and/or a surgical implant of the present invention, for
regenerative medicine and tissue regeneration or tissue replacement,
   e.g. regeneration of adipose and cartilage tissue,
implantable scaffold
disease model
wound treatment and/or wound healing,
2D and 3D synthetic cell culture substrate,
stem cell therapy,
drug delivery, preferably sustained or controlled release drug delivery
injectable therapies,
treatment of degenerative diseases of the skeletal system,
   e.g. degenerative disc disease, or urinary incontinence
biosensor development,
high-throughput screening,
biofunctionalized surfaces,
biofabrication, such as bio-printing,
cosmetic use;
and
gene therapy.

In accordance with an aspect of the present invention, the invention provides a method of tissue regeneration or tissue replacement comprising the steps:
a) providing a hydrogel according to the present invention, or a co-gel or co-hydrogel according to the present invention;
b) exposing said hydrogel or co-gel or co-hydrogel to cells which are to form regenerated tissue;
c) allowing said cells to grow on or in said hydrogel.

In one embodiment, the method is performed in vitro or in vivo or ex vivo.

Preferably, the method is performed in vivo, wherein, in step a), said hydrogel or co-gel or co-hydrogel is provided at a place in the body of a patient where tissue regeneration or tissue replacement is intended.

In one embodiment, said step a) is performed by injecting said or co-gel or co-hydrogel or a solution of at least one depsipeptide of the present invention, at a place in the body of a patient where tissue regeneration or tissue replacement is intended.

In one embodiment, said step a) further comprises the co-injection of a gelation enhancer, preferably of a solution of a salt.

Preferably, the method is performed ex vivo, wherein, in step a) or b), cells from a patient or from a donor are mixed with said hydrogel or co-gel or co-hydrogel, and the resulting mixture is provided at a place in the body of a patient where tissue regeneration or tissue replacement is intended.

In one embodiment, said tissue is selected from the group comprising skin tissue, nucleus pulposus in the intervertebral disc, cartilage tissue, synovial fluid and submucosal connective tissue in the bladder neck.

In one embodiment, said hydrogel or co-gel or co-hydrogel comprises one or more bioactive therapeutics that stimulate regenerative processes and/or modulate the immune response.

This disclosure describes for the first time the ability of ultrashort aliphatic depsipeptides to self-assemble in water to form hydrogels. During the process of self-assembly, the peptides adopt a variety of different secondary structures that can be detected using circular dichroism. The depsipeptides can undergo hydrolysis, yielding fragments that do not form hydrogels alone or in combination. The chosen hexameric depsipeptide example shows good biocompatibility, and thus can be used in a biological setting.

Ultrashort aliphatic depsipeptides are biodegradable, which allows the gel to dissolve over time. They are thus ideal for in vivo applications where drug and gene delivery is needed and where it is not required that the scaffold exists over long time.

The hydrogels can serve to deliver α-hydroxy acids in a sustained fashion for topical cosmetic applications.

The bioactive α-hydroxy acid moiety can be carefully selected to exert biological/biochemical effects on cells/tissues (e.g. exfoliation). The depsipeptide can also be mixed with the parent ultrashort peptide to create co-gels, whose bulk stability and biodegradation rate can be controlled by relative composition of constituents. The hydrolysis of the ester bond of the depsipeptides would lead to 2 smaller fragments which can readily diffuse away, thereby reducing the total volume of the system and increasing the porosity of the bulk scaffold. For tissue engineering applications, this is a good strategy to enhance cell migration into the interior of the hydrogel over time, and allow for faster matrix remodeling via cellular secretion of extracellular matrix.

Stimuli-responsive nature of the depsipeptides opens avenues for applications in injectable therapies, bio-printing, and cell encapsulation. Since the depsipeptides demonstrate good solubility in water and form solutions with low viscosity, the solution will not clog the needle/printer. Upon interacting with a physiological salt solution (such as phosphate buffered saline, PBS), gelation occurs. The kinetics of gelation can be tuned by depsipeptide concentration, pH and ionic concentration.

In view of the stability of the parent peptide, the potential ability of the depsipeptide to dissociate the bulk hydrogel can be applied to gently release cells from 3D culture. The depsipeptide can also be used to de-stabilize the parent peptide hydrogels to reverse errors in application (particularly important for cosmetic applications, such as dermal fillers, whereby the patient may want to reduce the fullness of the treatment subsequently).

Other aspects and features of the present invention will become apparent to those skilled in the art upon review of the following description of specific embodiments of the invention in conjunction with the accompanying figures.

### BRIEF DESCRIPTION OF DRAWINGS

Embodiments of the invention will now be described by way of example with reference to the accompanying drawings.

FIG. 1: Synthetic scheme of the key step in the synthesis as well as the chemical structure of Ac-ILVaGK-NH₂.

FIG. 2: Optical photographs of two depsipeptide hydrogels in MilliQ water and 1× PBS buffer solution and the decomposed hydrogel.

FIG. 3: Rheological characterization of the depsipeptide.

FIG. 4: Morphology of Ac-ILVaGK-NH₂ at two different magnifications.

FIG. 5: Concentration dependent CD-spectra of Ac-ILVaGK-NH₂.

FIG. 6: Concentration effect curve of Ac-ILVaGK-NH₂ and its degradation products Ac-ILV-OH and HO-aGK-NH₂ in human mesenchymal stem cells.

Other arrangements of the invention are possible and, consequently, the accompanying drawings are not to be understood as superseding the generality of the preceding description of the invention.

### DETAILED DESCRIPTION

We have previously described ultrashort peptide sequences β-7 residues) which have an innate tendency to self-assemble into helical fibers that ultimately result in hydrogel formation, see e.g. WO 2011/123061, US 2014/0093473 A1, WO 2014/104981 A1 of the inventors.

The microarchitecture of these nanofibrous hydrogels resemble the extracellular matrix, opening avenues for widespread applications as biomimetic scaffolds for tissue engineering and three-dimensional cell culture. Furthermore, the ultrashort peptide hydrogels demonstrate remarkable mechanical stiffness, thermostability, and biocompatibility, in vitro and in vivo stability. In particular, the stability of these hydrogels offer attractive advantages to applications such as developing injectable therapies, such as for degenerative disc disease, as well as other tissue engineering applications requiring the construct to provide structural support over long durations.

However, in developing these hydrogels for applications such as injectable matrices for drug and gene delivery, where a fast delivery of the compounds is needed, it is desirable to increase the degradability of the hydrogel matrix. In such instances, a self-assembling hydrogel consists of well-defined constituents susceptible to biodegradation.

This disclosure describes a novel class of self-assembling aliphatic depsipeptides. Depsipeptides are peptides in which one or more of the amide groups (—C(O)NHR—) are replaced by the corresponding ester, —C(O)OR. Inspired by the structure of previously mentioned class of ultrashort self-assembling peptides, these depsipeptides differ in structure in that one of the amino acid constituents is substituted by an α-hydroxy acid with a similar structure. In doing so, we introduce an ester bond in place of an amide bond.

Ester bonds are more susceptible to hydrolysis and enzyme degradation in a biological context, enabling us to increase biodegradability and decrease the stability of the bulk hydrogel over time. The concept of substituting the amid bond by an ester bond can be used to investigate the importance of backbone hydrogen bonding since the ester bond lacks a proton, which in an ordinary peptide is a potential hydrogen bonding side. At the same time, the ester bond can be well compared with the amide bond with regards to torsion angle, bond anglel and bond length.

In addition, many α-hydroxy acids have demonstrated bioactive properties. Coupling them to a self-assembling peptidic backbone provides sustained delivery of the bioactive α-hydroxy acid.

The depsipeptides of the invention form hydrogels in aqueous conditions, via a different mechanism than reported by Nguyen et al. (2014) or in WO 2010/019716 A1. The design is inspired by the above mentioned class of self-assembling ultrashort peptides. The characteristic motif that drives the self-assembly of the parent peptides consists of a N-terminus “tail” of 2 to 7 natural aliphatic amino acids, arranged in overall decreasing hydrophobicity towards the C-terminus with the hydrophilic C-terminal amino acid forming a polar “head”. Self-assembly in aqueous conditions occurs when the amino acids pair and subsequently stack in antiparallel fashion on top of each other to form helical fibers. Hydrogels are obtained upon further aggregation of the fibrils into 3D nanofibrous networks that entrap water (Mishra et al., 2011; Reithofer et al., 2014-a; Reithofer et al., 2014-b; Hauser et al., 2011).

In designing the depsipeptides, one of the constituent aliphatic amino acids will be replaced by an α-hydroxy acid with a similar structure. As for the example of the depsipeptide analog of Ac-ILVAGK-NH₂, alanine (A) was replaced with lactic acid (Ac-ILVaGK-NH₂ with a=lactic acid; SEQ ID NO. 52).

TABLE 1
Embodiments of depsipeptide sequences

| N- | Peptide | C- | α-hydroxy | SEQ ID |
|---|---|---|---|---|
| Terminus | sequence | Terminus | acid | NO. |
| Ac | LIVAgK | NH₂ | glycolic acid | 49 |
| Ac | LIVaGK | NH₂ | lactic acid | 50 |
| Ac | ILVAgK | NH₂ | glycolic acid | 51 |
| Ac | ILVaGK | NH₂ | lactic acid | 52 |
| Ac | IVLAgK | NH₂ | glycolic acid | 53 |
| Ac | IVLaGK | NH₂ | lactic acid | 54 |
| Ac | LIVAgD | OH | glycolic acid | 55 |
| Ac | LIVaGD | OH | lactic acid | 56 |
| Ac | AIVAgS | OH | glycolic acid | 57 |
| Ac | AIVaGS | OH | lactic acid | 58 |
| Ac | IVDa | OH | lactic acid | 69 |
| Ac | IVaD | OH | lactic acid | 59 |
| Ac | IVa | OH | lactic acid | |
| Ac | ILa | OH | lactic acid | |
| Ac | IVKa | NH₂ | lactic acid | 70 |

Despite changing one of the amide bonds to an ester linkage, the resulting depsipeptides are still capable to self-assemble into hydrogels. However, a significantly higher concentration of starting material is required when compared to the parent peptide. Interestingly, CD studies revealed that in the process of self-assembly, the depsipeptides adopt two different intermediate secondary structures before attaining the final β-turn structure. With increasing depsipeptide concentration, first α-helical and then β-sheet intermediate structure were detected, before the final β-turn structure was reached.

We also investigated the degradation, mechanical properties and cytocompatibility of the depsipeptide hydrogels. Degradation studies revealed that the depsipeptides showed a pH dependent degradation, where hydrolysis can be accelerated at basic conditions. Although the depsipeptides formed stiff hydrogels, a significantly higher concentration was required to attain the comparable rigidity to the parent peptide. The depsipeptides and the degradation products of ester hydrolysis were cytocompatible. This bodes well to their use in biomedical applications as scaffolds for tissue engineering and matrices for drug delivery.

### EXAMPLES

1. Materials and Methods

1.1 Materials

Fmoc-lys-rink resin (resin 0.42 mg/mol), Fmoc protected amino acid i.e. glycine, alanine, valine, leucine and isoleucine, 2-(7-Aza-1H-benzotriazole-1-yl)-1,1,3,3 -tetramethyluronium hexafluorophosphate) (HATU) were purchased from GL Biochem (Shanghai) Ltd. Dimethylformamide (DMF) (analytical grade) was purchased from Fisher Scientific UK. Acetic anhydride (Ac₂O) and dimethyl sulfoxide (DMSO) was purchased from Sigma Aldrich. N,N-Diisopropylethylamine (DIPEA), dichloromethane (DCM), trifluoroacetic acid (TFA) and TIS (triisopropylsilane) were purchased from Alfa Aesar, a Johnson Matthey Company. Piperidine was purchased from Merck Schuchardt OHG. Diethyl ether (Et₂O) was purchased from Tedia Company Inc. and lactic acid was purchased from Sigma-Aldrich. All chemicals were used as received.

All peptide based compounds were purified on an Agilent 1260 Infinity preparative HPLC system equipped with a phenomenex Lunar C18 column (150×21.2 mm 5 μM). The HPLC was coupled over an active splitter to a SQ-MS for mass triggered fraction collection. MilliQ water and HPLC grade acetonitrile, both containing 0.1% formic acid, were used as eluents. ¹H and ¹³C NMR spectra were recorded on a Bruker AV-400 (400 MHz) instrument and all signals were referenced to the solvent residual peak.

1.2 Depsipeptide Preparation

Acetylated isoleucine-leucine-valine-lactic acid-glycine-lysine (Ac-ILVaGK-NH₂, a=lactic acid) [SEQ ID NO: 52] depsipeptide was synthesised using solid phase peptide synthesis method.

Briefly, Fmoc-lys-rink resin was weighed out and swelled for one hour using DMF. Afterwards, 10 equivalents of Ac₂O and DIPEA was added to block any free amine on the resin, and allowed to react for 45 minutes. The resin was then washed with DMF before going through a series of de-protection reactions using 20% piperidine in DMF and coupling reactions with the addition of the 3 equivalent of desired amino acid with TBTU in the presence of HOBT and DIPEA. After the coupling of Fmoc-Gly-OH the Fmoc group was removed and lactic acid (3 equiv.) was coupled using TBTU, HOBT and DIPEA as coupling reagent. The reaction was allowed to proceed for 10 minutes. After washing the resin 5 times DMF and twice with DCM, the esterification was performed using 8 equivalents of Fmoc-Val-OH, DIC and 10 mol % of DMAP. For this purpose, Fmoc-Val-OH was dissolved in DMF/DMC (2:1) and DIC and DMAP were added. The reaction was allowed to proceed overnight. The following amino acids were coupled as described above using TBTU as coupling reagent.

Acetylation of the N-terminus was performed using 4 time excess of Ac₂O and DIPEA. Following all reactions, the resins was washed with DMF and DCM, allowed to dry before cleaving the peptide from the resins with a mixture of 95% TFA, 2.5% water and 2.5% TIS. The solvents were removed under reduced pressure and Et₂O was later added to precipitate the peptide. The peptide was isolated by centrifugation, washed twice with Et₂O and dried under reduced pressure. Purification was performed in a preparative high-performance liquid chromatography electronspray ionization mass spectroscopy (HPLC ESI MS) (purchased from Agilent Technologies, 1260 infinity series) HPLC-MS system by dissolving the peptide in a minimum amount of DMSO. Yield: 1.28 g (60%).

¹H-NMR (d₆-dmso): 8.23 (m, 1H), 8.03, (m, 2H), 7.91 (m, 2H), 7.37 (s, 1H), 7.08 (s, 1H), 4.98 (m, 1H), 4.38 (m, 1H), 4.26 - 4.11 (m, 3H), 3.78 (m, 2H), 2.74 (m, 2H), 2.10 (m, 1H), 1.84 (s, 3H), 1.68 (m, 2H), 1.62-1.20 (m, 12H), 1.06 (m, 1H), 0.88 (m, 9H), 0.80 (m, 9H) ppm.

¹³C-NMR (d₆-dmso): 173.4, 172.4, 171.1, 170.5, 170.2, 169.3, 168.3, 70.1, 57.1, 56.9, 52.0, 50.8, 41.8, 40.7, 38.8, 36.8, 31.5, 29.8, 26.8, 24.4, 24.1, 23.0, 22.5, 22.3, 21.7, 18.9, 17.9, 17.7, 15.4, 11.0 ppm.

ESI-MS: Calculated for C₃₀H₅₆N₇O₈ ([M+H³⁰ ]⁺)642.42, Found: m/z 62.4.

1.3 Synthesis of Ac-ILV-OH:

The peptide was synthesised similar to the method described above for standard Fmoc based synthesis. However, the rink amide resin was replaced with Wang resin in order to yield the unprotected peptide. Cleaving and purification was done as described above.

¹H-NMR (d₆-dmso): 12.59 (bs, 1H), 8.03 (d, ³J_{II,II}=8.4 Hz, 1H), 7.92 (d, ³J_{II,II}=8.8 Hz, 1H), 7.78 (d, ³J_{H,H}=8.5 Hz, 1H),4.37 (m, 1H) 4.14 (m, 2H), 2.04 (m, 1H), 1.84 (s, 3H), 1.68 (m, 1H), 1.59 (m, 1H), 1.42 (m, 3H) 1.06 (m, 1H), 0.90-0.75 (m, 18H) ppm

¹³C-NMR(d₆-dmso):173.2,172.4, 171.5, 169.6, 57.5, 57.2, 51.3, 41.2, 36.9, 30.3, 24.8, 24.5, 23.5, 22.9, 22.1, 19.5, 18.3, 15.8, 11.4 ppm

ESI-MS: Calculated for C₁₉H₃₆N₃O₅ ([M+H⁺]⁺) 386.27, Found: m/z 386.2.

1.4 Synthesis of HO-aGK-NH:

The peptide was synthesized similar to the method described above; however, the peptide was cleaved after lactic acid was coupled.

¹³C-NMR (H₂O/D₂O 95:5): 178.4, 176.7, 171.5, 67.6, 53.2, 42.1, 39.3, 30.3, 26.2, 21.9, 19.5 ppm.

ESI-MS: Calculated for C₁₁H₂₃N₄O₄ ([M+H⁺]⁺) 275.17, Found: m/z 275.2.

1.5 Oscillatory Rheometry

Rheometry was performed on TA ARES-G2 Serial at 27° C. on a 8 mm parallel plate serrated stainless steel. Frequency sweeps were performed at 0.1% strain from 0.1 rad/s to 100 rad/s/Strain sweeps were performed at 1 rad/s from 0.01 to 100% strain.

1.6 CD-Spectroscopy

CD spectra were collected with an Aviv 410 CD spectrophotometer fitted with a Peltier temperature controller, using a rectangular quartz cuvette with a fitted cap and an optical path length of 0.01. Data acquisition was performed in steps of 0.5 nm at a wavelength range from 190-270 nm.

1.7 FESEM

Hydrogel samples were shock frozen and kept at −80° C. Frozen samples were then freeze-dried. Lyophilized samples were fixed onto a sample holder using a carbon conductive tape and sputtered with platinum from both the top and the sides in a JEOL JFC-1600 High Resolution Sputter Coater. The coating current was 20 mA and the process lasted for 50 sec. The surface of interest was then examined with a JEOL JSM-7400F Field Emission Scanning Electron Microscopy (FESEM) system using an accelerating voltage of 2 kV.

1.8 Cytocompatibility

Human mesenchymal stem cells, hMSCs, were obtained from Lonza (Basel, Switzerland) and cultured in MSC growth medium (MSCGM) with 5% fetal bovine serum, 2% L-glutamine and 0.1% penicillin-streptomycin (Lonza). The cell metabolic activity was measured with the Cell Proliferation Reagent WST-1 assay (Roche Diagnostics, Mannheim, Germany). Briefly, 5000 hMSC cells were seeded in each well and media containing the sample was added to the desired concentration (n=6). After incubation for 72 hours at 37° C., the media was aspirated and media containing 10% WST-1 reagent added for 2 hours. The absorbance at 450 nm was measured subtracting the absorbance at 600 nm. The absorbance readings were further normalized against cells cultured in media containing an equivalent volume of PBS (i.e. 100% cell survival) to determine the percentage cell viability. Cells cultured with an equivalent volume of ethanol were used as a negative control (i.e. 100% cell death).

2. Results and Discussion

2.1 Design and Synthesis

As discussed above, we have previously reported a new class of aliphatic amphiphilic ultrashort peptides which have an innate tendency to self-assemble in water to form biomimetic, nanofibrous hydrogels with very high mechanical strength and are extremely stable in vitro and in vivo. Their stability and resistance towards fast enzymatic degradation has been an advantage for applications such as injectable therapies for degenerative disc disease and other tissue engineering applications requiring the construct to provide structural support over long durations. However, in developing these hydrogels where the hydrogel needs to degrade in rapid manner, such as injectable matrices for drug and gene delivery, a self-assembling hydrogel with enhanced degradation would be advantageous.

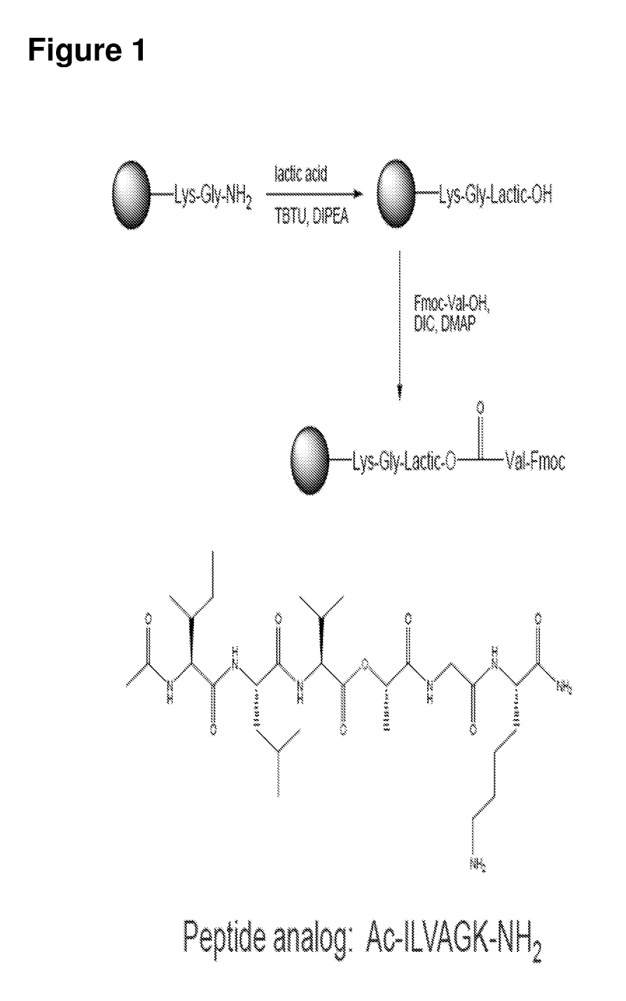

In order to design hydrolytically active depsipeptide analogues, we replaced one amino acid in the peptide sequence with an α-hydroxy acid. As one embodiment, the depsipeptide analogue of Ac-ILVAGK-NH₂ (Ac-IK₆-NH₂) [SEQ ID NO: 71] was synthesised wherein alanine (A) was replaced with lactic acid (Ac-ILVaGK-NH₂; a=lactic acid, FIG. 1) [SEQ ID NO: 52]. The synthesis was carried out on solid phase using Fmoc-Lys-Rink-Am resin as the starting material. Standard peptide synthesis protocols were used; however the esterification reaction was carried out overnight using DIC/DMAP as the coupling reagent. The final depsipeptide was obtained in good yield after HPLC purification.

2.2 Gelation properties

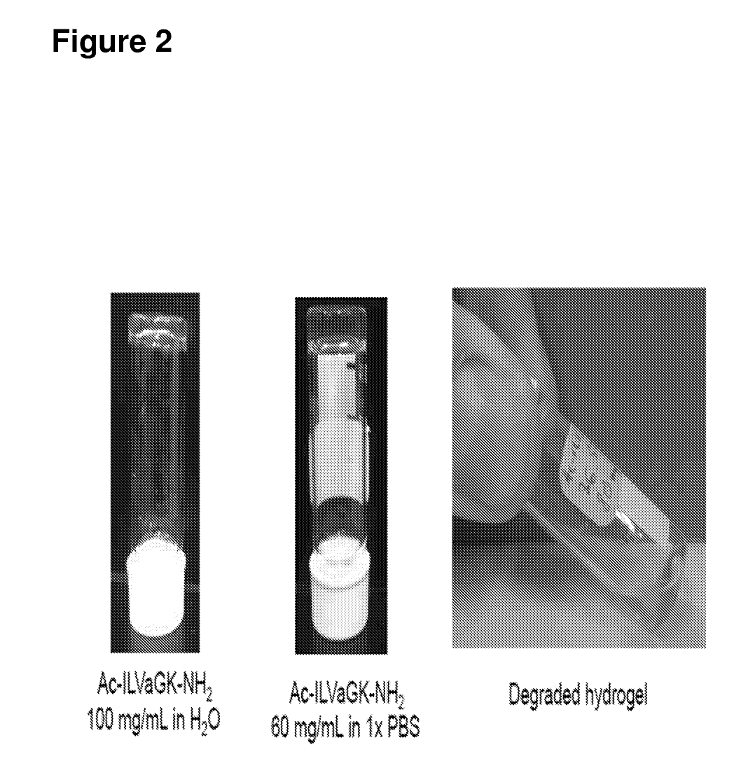

In order to determine the minimum gelation concentration in water, the depsipeptide was dissolved in MilliQ water. Based on our experience with the parent peptide Ac-IK₆-NH₂, which has a minimum gelation concentration of 10 mg/mL in MilliQ water, we expected similar results for the depsipeptide. However, a hydrogel formation could only be observed at a concentration of 100 mg/mL when the sample was left at room temperature. In contrast, when a sample containing 100 mg/mL depsipeptide was dissolved in 90% of water and 10% 10xPBS was added, instantaneous hydrogel formation was observed. This observation corroborates with the known stimuli-enhanced gelation of Ac-IK₆-NH₂ in the presence of a salt solution. Furthermore, by adding PBS buffer the minimum gelation concentration could be lowered to 60 mg/mL (FIG. 2).

In general, the minimum gelation concentration of Ac-ILVaGK-NH₂ [SEQ ID NO: 52] in water as well as in 1xPBS buffer is about 10 times higher than its parent peptide Ac-IK₆-NH₂ [SEQ ID NO: 71]. This result suggests that replacing one of the amide bonds, with an ester bond significantly changes the ability of the peptide to from stable aggregates in water and also demonstrates the importance of hydrogen bonding in the self-assembling process. While an ester bond has comparable bond angles to the amide bond, it could act as a hydrogen bond acceptor but is unable to be a proton donor. This could explain the higher minimum gelation concentrations. This behaviour is in accordance to observations by Liskamp and co-workers (Rijkers et al., 2002). Liskamp reported depsipetides based on the amylin (20-29) structure. It was shown, that the replacement of a key residue with an ester bond was sufficient to delay gelation significantly and inhibit fiber formation (Rijkers et al., 2002).

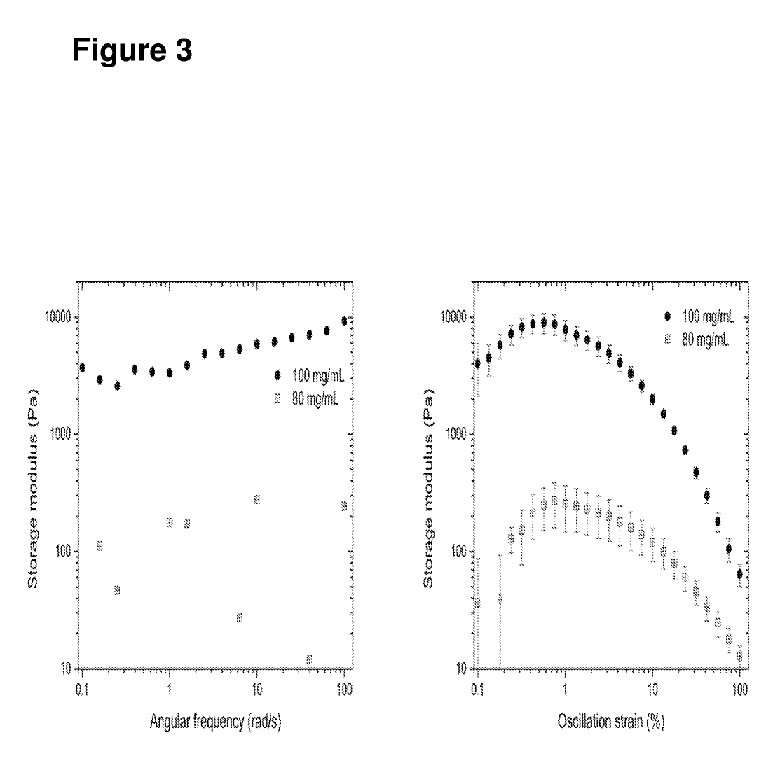

Although the depsipeptides can form degradable hydrogels by them self, they can be added to destabilize the bulk hydrogel structure of their parent ultrashort peptides. In view of the stability of the parent peptide, the potential ability of the depsipeptide to dissociate the bulk hydrogel can be applied to gently release cells from 3D culture. The depsipeptide can be used to de-stabilise the parent peptide hydrogels. This is advantageous where application errors should be reversed (particularly important for cosmetic applications such as dermal fillers where the patient may want to reduce the fullness of the treatment subsequently). The mechanical properties support this claim (see FIG. 3).

We can form co-gels of the depsipeptide and parent peptide where the degradation rate would depend on the relative concentrations of its constituents. In such co-gel systems, the hydrolysis of the ester bond leads to 2 smaller fragments which can readily diffuse away, thereby reducing the total volume of the system and increasing the porosity of the bulk scaffold. For tissue engineering applications, this provides favorable strategy to enhance cell migration into the interior of the hydrogel over time and allows for natural matrix remodeling via cellular secretion of extracellular matrix.

The stimuli-responsive nature of the depsipeptides opens avenues for applications in injectable therapies, bio-printing, and cell encapsulation. Since the depsipeptides demonstrate good solubility in water, forming solutions with low viscosity, will prevent a clogging of the solution in the needle/printer. The interacting with a physiological salt solution (such as phosphate buffered saline, PBS), stimulates gelation. The kinetics of gelation can be tuned by depsipeptide concentration, pH and ionic concentration. We can encapsulate cells, nanoparticles, small molecules and therapeutic drugs, oligonucleotides, nucleic acids and proteins during gelation.

Extending the technology towards 3D microdroplet printing and bio-moulding, we can obtain biological, organotypic constructs with distinct, multi-functional micro-niches.

Multi-cellular constructs can also be obtained as the hydrogel can spatially confine different cell types during the printing process. The scaffold will provide the co-encapsulated cells with mechanical stability. Genes, molecules, growth factors and other proteins can be co-delivered to enhance cell survival, promote stem cell differentiation and modulate the host immune response. The resulting 3D biological constructs can be used as organoid models for screening drugs, studying cell behaviour and disease progression, as well as tissue-engineered implants for regenerative medicine.

2.3 FESEM Study

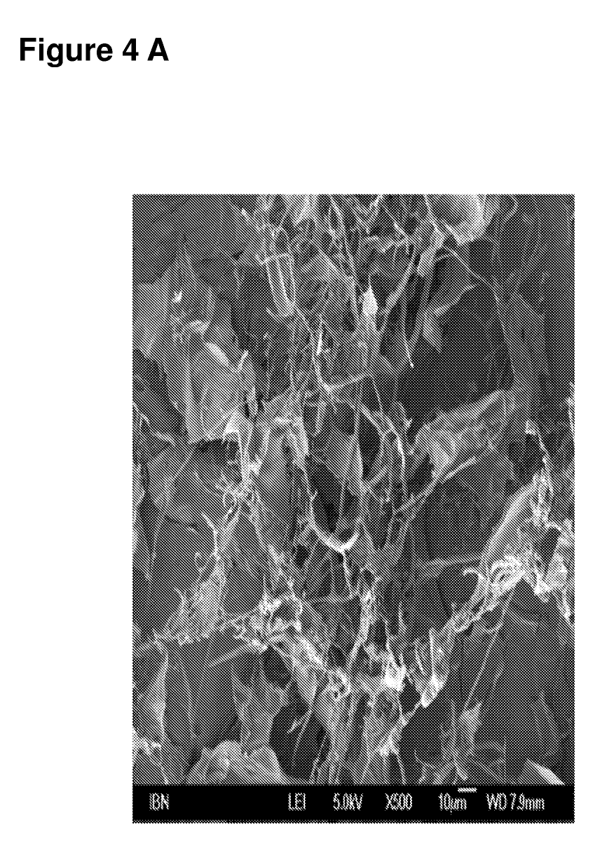

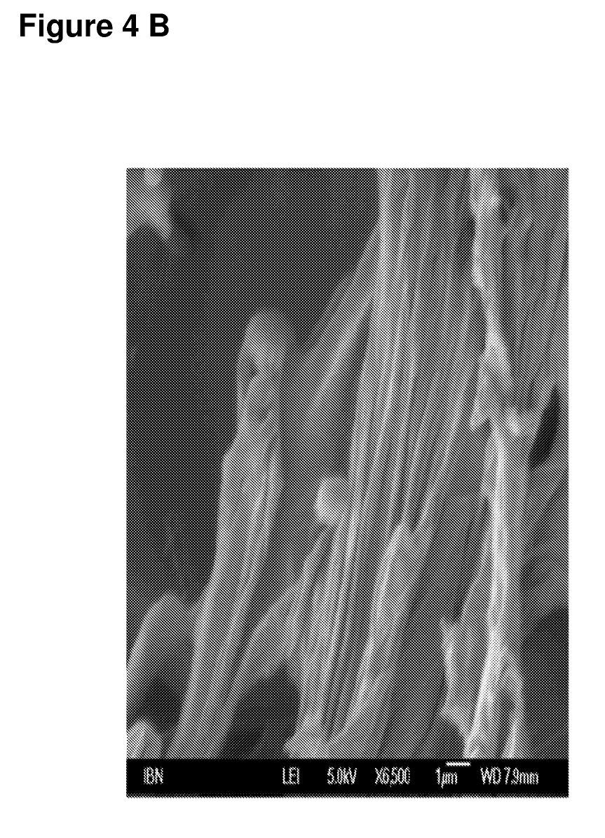

Morphological characterization of the depsipeptide hydrogel scaffolds was done by Field Emission Scanning Electron Microscopy (FESEM) and representative images for a hydrogel of Ac-ILVaGK-NH₂ [SEQ ID NO: 52] are shown in FIG. 4. A fibrillization of the depsipeptide is clearly visible in both images, confirming the ability of the compound to self-assemble in water.

2.4 CD spectroscopy

To further characterize the influence of the ester bond CD studies were carried out. We have previously reported on the self-assembling process of this class of ultrashort peptides (Mishra et al., 2011; Hauser et al., 2011). Detailed CD studies demonstrated concentration dependent changes in the secondary structure. At low concentration, the ultrashort self-assembling parent peptide displays a random coil structure, which changes into an α-helical secondary structure with increase in concentration. If the concentration is further increased a second transition to a β-turn structure is observed, which can also be seen as the final or stabile state.

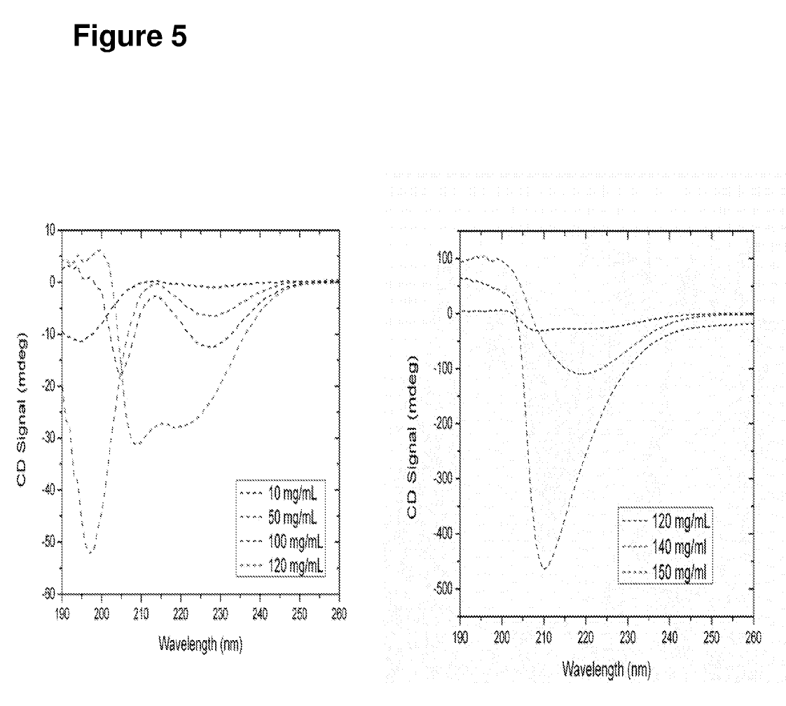

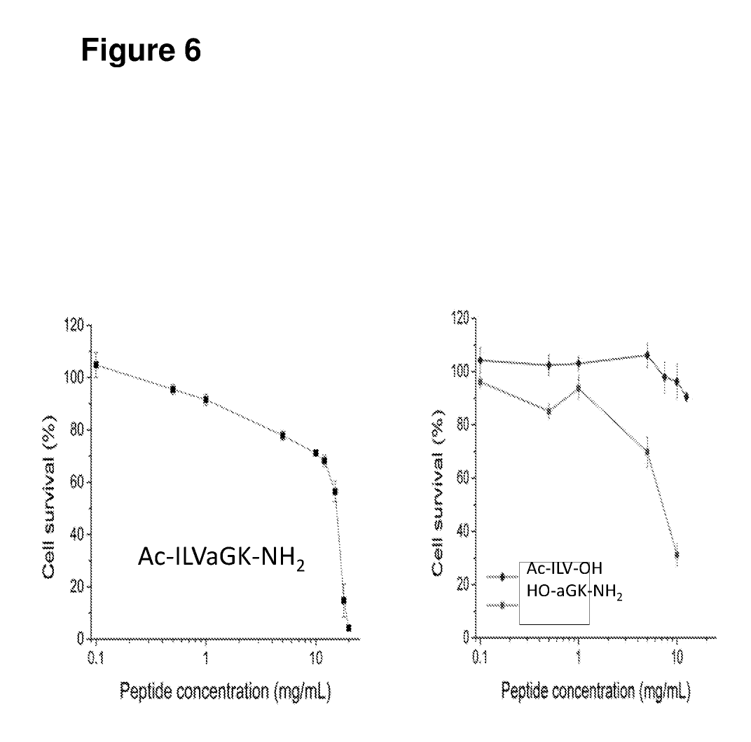

When the depsipeptide was investigated by CD spectroscopy, a slightly different transition to the final structure was observed. Different concentrations of depsipeptide were freshly prepared and CD spectra were recorded. As expected, the depsipeptide Ac-ILVaGK-NH₂ [SEQ ID NO. 52] displayed concentration-dependent changes in the secondary structure. The results are shown in FIG. 5. In accordance with the observations made with the parent peptide, the depsipeptide displayed a random coil structure at lower concentration. However, in comparison to the parent peptide, the random coil structure is prevalent up to high concentrations of more than 50 mg/mL. Only at 100 mg/mL a transition to an α-helix starts to get established. At 120 mg/mL, a well-defined α-helical conformation is observed. Interestingly, when the concentration was further increased, a second intermediate structure was detected. In contrast to Ac-IK₆-NH₂ [SEQ ID NO: 71], which displayed a conformation change from α-helix to β-turn, the depsipeptide displayed a change from α-helix to β-sheet.

On the contrary, when investigating the CD spectra of the parent ultrashort self-assembling peptides, a β-sheet structure was never observed.

So far, a β-sheet structure was never observed by CD-spectroscopy in our class of ultrashort peptides. To determine, if the β-sheet confirmation represents the final state of assembly, the concentration of the solution was increased to 150 mg/mL. The resulting CD spectrum displayed a further change in structural confirmation, whereby a transition from β-sheet to β-turn could be observed.

To the best of our knowledge, this is the first example of a peptide/depsipeptide which undergoes three concentration-dependent changes of its secondary structure. The results indicate that the β-sheet structure is a very labile structure and can better be described as a snap shot of an α-helix to β-turn conformation change which passes through a β-sheet transition. The exchange of the amide to ester bond appears to significantly slow down and reduce the speed of α-helix to β-turn transition. In addition, the stability of the final β-turn structure seems to be closer to the β-sheet transition state which leads to an observable β-sheet confirmation by CD spectroscopy. Based on these results, we believe, that most of our peptides undergo an α-helix to β-sheet to β-turn transition, which cannot be observed in a CD spectrum due to the significantly higher stability of the β-turn structure compared to the β-sheet structure. By removing one hydrogen bond donor, the stability of the final structure seems to be reduced, which can also be seen by the increase in minimum gelation concentration, and thus leads to an observable β-sheet structure.

2.5 Degradation studies

In order to test the degradation ability of the depsipeptide, 2 mg/mL was dissolved in PBS puffer at pH 5.7, 7.3 and 8.5. All samples were incubated at 37° C. and analysed by HPLC-MS. A pH dependence was observed with the depsipeptide being least stable at pH 8.5 and the most at pH 5.7.

In order to characterize the degradation products of the depsipeptide Ac-ILV-OH and HO-aGK-NH2 were synthesized and the retention time was determined by HPLC-MS. As expected the degradation products of the depsipeptide showed not only the same retention time, but the identity of the compound was also confirmed by its mass spectrum.

2.6 Cytocompatibility

In order to determine depsipeptide cytotoxicity and effect on cell proliferation, human mesenchymal stem cells (hMSCs) were incubated with various concentrations of the depsipeptide and their metabolic activity and viability was tested after 3 days. Cell viability decreased with increasing depsipeptide concentration. The IC50 value was approximately 15 mg/mL. To address concerns that the cytotoxicity could be attributed to the degradation products following hydrolysis of the ester bond, the possible degradation products, the peptide Ac-ILV and depsipeptide HO-aGK-NH₂, were also evaluated. The peptidic fragments were extremely well tolerated, particularly, if the acidity was neutralized. The depsipeptide fragment demonstrated significant cytotoxicity at concentrations exceeding around 10 mg/mL.

2.7 Conclusion

We report here the synthesis of depsipeptides which is derived from a class of ultrashort aliphatic peptides. In comparison with their parent peptides could have up to 10 fold increase in the minimum gelation concentration. They display stimuli responsiveness to salt, which reduces the amount of depsipeptide needed for hydrogel formation by almost 50%. CD studies, revealed, that the depsipeptide displays transitions in the secondary structure showing conformational changes from random coil to α-helix to β-sheet and β-turn structures. Degradation studies showed that the depsipeptide can undergo hydrolysis at the ester bond, and the rate of degradation was found to be pH dependent.

It is to be understood that the described embodiment(s) have been provided only by way of exemplification of this invention, and that further modifications and improvements thereto, as would be apparent to persons skilled in the relevant art, are deemed to fall within the broad scope and ambit of the present invention described herein.

### REFERENCES

Y. Chau, Y. Luo, A. C. Y. Cheung, Y. Nagai, S. Zhang, J. B. Kobler, S. M. Zeitels and R. Langer, Biomaterials, 2008, 29, 1713-1719.

L. E. Freed, G. Vunjak-Novakovic, R. J. Biron, D. B. Eagles, D. C. Lesnoy, S. K. Barlow and R. Langer, Nat Biotech, 1994, 12, 689-693.

K. M. Galler, L. Aulisa, K. R. Regan, R. N. D′ Souza and J. D. Hartgerink, Journal of the American Chemical Society, 2010, 132, 3217-3223.

M. C. Giano, D. J. Pochan and J. P. Schneider, Biomaterials, 2011, 32, 6471-6477.

C. A. E. Hauser, R. Deng, A. Mishra, Y. Loo, U. Khoe, F. Zhuang, D. W. Cheong, A. Accardo, M. B. Sullivan, C. Riekel, J. Y. Ying and U. A. Hauser, Proceedings of the National Academy of Sciences, 2011, 108, 1361-1366.

S. L. Ishaug, G. M. Crane, M. J. Miller, A. W. Yasko, M. J. Yaszemski and A. G. Mikos, Journal of Biomedical Materials Research, 1997, 36, 17-28.

H. W. Jun, V. Yuwono, S. E. Paramonov and J. D. Hartgerink, Advanced Materials, 2005, 17, 2612-2617.

B.-S. Kim and D. J. Mooney, Trends in Biotechnology, 1998, 16, 224-230.

Y. Kumada, N. A. Hammond and S. Zhang, Soft Matter, 2010, 6, 5073-5079.

Y. Loo, Y.-C. Wong, E. Z. Cai, C.-H. Ang, A. Raju, A. Lakshmanan, A. G. Koh, H. J. Zhou, T.-C. Lim, S. M. Moochhala and C. A. E. Hauser, Biomaterials, 2014, 35, 4805-4814.

D. M. Lynn, M. M. Amiji and R. Langer, Angewandte Chemie International Edition, 2001, 40, 1707-1710.

A. Mishra, Y. Loo, R. Deng, Y. J. Chuah, H. T. Hee, J. Y. Ying and C. A. E. Hauser, Nano Today, 2011, 6, 232-239.

M. M. Nguyen, K. M. Eckes and L. J. Suggs, Soft Matter, 2014, 10, 2693-2702.

M. R. Reithofer, K.-H. Chan, A. Lakshmanan, D. H. Lam, A. Mishra, B. Gopalan, M. Joshi, S. Wang and C. A. E. Hauser, Chemical Science, 2014, 5, 625-630. (Reithofer et al., 2014-a)

M. R. Reithofer, A. Lakshmanan, A. T. K. Ping, J. M. Chin and C. A. E. Hauser, Biomaterials, 2014, 35, 7535-7542. (Reithofer et al., 2014-b)

D. T. S. Rijkers, J. W. M. Hoppener, G. Posthuma, C. J. M. Lips and R. M. J. Liskamp, Chemistry—A European Journal, 2002, 8, 4285-4291.

E. Ron, T. Turek, E. Mathiowitz, M. Chasin, M. Hageman and R. Langer, Proceedings of the National Academy of Sciences, 1993, 90, 4176-4180.

Y. F. Tian, G. A. Hudalla, H. Han and J. H. Collier, Biomaterials Science, 2013, 1, 1037-1045.

S. Zhang, T. Holmes, C. Lockshin and A. Rich, Proceedings of the National Academy of Sciences, 1993, 90, 3334-3338.

1. A depsipeptide, capable of self-assembling and forming a hydrogel, having the general formula selected from:
Zₐ-(X^{AHA})_{b}-(Y)_{c}-Z′_{d} (I)
wherein
Z is an N-terminal protecting group;
a is at least 1,
X^{AHA} is, at each occurrence, independently selected from the group consisting of aliphatic amino acids and aliphatic amino acid derivatives and includes at least one α-hydroxy acid, wherein the α-hydroxy acid is at least one selected from the group consisting of lactic acid, glycolic acid, malic acid, 2,3-dihydroxypropanoic acid, lactobionic acid, and citric acid;
b is an integer selected from 2 to 7;
Y is selected from the group consisting of polar amino acids and polar amino acid derivatives;
c is 0, 1 or 2;
Z′ is a C-terminal protecting group; and
d is 0 or 1, and
b+c is at least 3;
Zₐ-(X)_{b1}-(AHA)_{d}-(X)_{b2}-(Y)_{c}-Z′ₑ (II)
wherein
Z is an N-terminal protecting group;
a is at least 1;
X is, at each occurrence, independently selected from the group consisting of aliphatic amino acids and aliphatic amino acid derivatives;
with each of bl and b2 being an integer selected from 0 to 7, under the proviso that bl +b2 is 2 to 7,
AHA is, at each occurrence, independently selected from the group consisting of α-hydroxy acids, wherein the α-hydroxy acid is at least one selected from the group consisting of lactic acid, glycolic acid, malic acid, 2,3-dihydroxypropanoic acid, lactobionic acid, and citric acid;
d is 1 or 2;
Y is selected from the group consisting of polar amino acids and polar amino acid derivatives;
c is 0, 1 or 2;
under the proviso that b1+b2+c+d<7,
Z′ is a C-terminal protecting group; and
e is 0 or 1;
Zₐ-(X)_{b′}-(Y)_{c}-(AHA)_{d}-Z′ₑ (III)
wherein
Z is an N-terminal protecting group;
a is at least 1;
X is, at each occurrence, independently selected from the group consisting of aliphatic amino acids and aliphatic amino acid derivatives;
b′ is an integer selected from 2 to 7;
Y is selected from the group consisting of polar amino acids and polar amino acid derivatives;
c is 0, 1 or 2;
AHA is, at each occurrence, independently selected from the group consisting of α-hydroxy acids, wherein the α-hydroxy acid is at least one selected from the group consisting of lactic acid, glycolic acid, malic acid, 2,3-dihydroxypropanoic acid, lactobionic acid, and citric acid;
d is 1 or 2;
Z′ is a C-terminal protecting group; and
e is 0 or 1, and
b′ +c is at least
wherein all or a portion of said aliphatic amino acids are arranged in an order of decreasing amino acid size in the direction from N- to C-terminus, wherein the size of the aliphatic amino acids is defined as I=L>V>A>G, and/or
wherein the overall hydrophobicity decreases from N- to C-terminus.

2.-5. (canceled)

6. The depsipeptide according to claim 1, wherein (X^{AHA})_{b} of formula I or (X)_{b1}-(AHA)_{d}-(X)_{b2} of formula II has a sequence selected from:
(SEQ ID NO: 1)
LIVAG(AHA),
(SEQ ID NO: 2)
LIVA(AHA),
(SEQ ID NO: 3)
LIV(AHA)G,
(SEQ ID NO: 4)
ILVAG(AHA),
(SEQ ID NO: 5)
ILVA(AHA),
(SEQ ID NO: 6)
ILV(AHA)G,
(SEQ ID NO: 7)
IVLA(AHA),
(SEQ ID NO: 8)
IVL(AHA)G,
(SEQ ID NO: 9)
AIVAG(AHA),
(SEQ ID NO: 10)
AIVA(AHA),
(SEQ ID NO: 11)
AIV(AHA)G,
IV(AHA),
IL(AHA),
wherein AHA refers to α-hydroxy acid.

7. The depsipeptide according to claim 6, wherein (X^{AHA})_{b} of formula I or (X)_{b1}-(AHA)_{d}-(X)_{b2} of formula II has a sequence selected from:
(SEQ ID NO: 12)
LIVAg,
(SEQ ID NO: 13)
LIVaG,
(SEQ ID NO: 14)
LIVAm,
(SEQ ID NO: 15)
LIVAGm,
(SEQ ID NO: 16)
ILVAg,
(SEQ ID NO: 17)
ILVaG,
(SEQ ID NO: 18)
ILVAm,
(SEQ ID NO: 19)
ILVAGm,
(SEQ ID NO: 20)
IVLAg,
(SEQ ID NO: 21)
IVLaG,
(SEQ ID NO: 22)
AIVAg,
(SEQ ID NO: 23)
AIVaG,
(SEQ ID NO: 24)
AIVAGm,
IVa,
ILa,
IVm,
wherein “g” refers to glycolic acid, “a” refers to lactic acid and “m” refers to malic acid.

8. The depsipeptide according to claim 1, wherein (X)_{b′} of formula III has a sequence selected from:
IV,
wherein (X)_{b′} of formula III has a sequence selected from:
(SEQ ID NO: 25)
LIVAG,
(SEQ ID NO: 26)
ILVAG,
(SEQ ID NO: 27)
LIVAA,
(SEQ ID NO: 28)
LAVAG,
(SEQ ID NO: 29)
AIVAG
(SEQ ID NO: 30)
GIVAG
(SEQ ID NO: 31)
VIVAG
(SEQ ID NO: 32)
ALVAG
(SEQ ID NO: 33)
GLVAG
(SEQ ID NO: 34)
VLVAG
(SEQ ID NO: 35)
IVAG
(SEQ ID NO: 36)
LIVA
(SEQ ID NO: 37)
LIVG
and
IVA-,

9. The depsipeptide according to claim 1, wherein
in formula I b is an integer from 2 to 7, preferably 3 to 7
in formula II b1+b2 is 2 to 7, prcfcrably 3 to 7 and
in formula III b′ is an integer from 2 to 7, preferably 3 to 7.

10. (canceled)

11. The depsipeptide according to claim 1,
wherein c is 2 and said polar amino acids are identical amino acids, or
wherein c is 1 and said polar polar amino acid comprises any one of aspartic acid, asparagine, glutamic acid, glutamine, serine, threonine, cysteine, methionine, lysine, ornithine, 2,4-diaminobutyric acid (Dab) and histidine.

12. The depsipeptide according to claim 1, wherein (Y)_{c} has a sequence selected from Asp, Asn, Glu, Gln, Ser, Thr, Cys, Met, Lys, Orn, Dab, His, Asn-Asn, Asp-Asp, Glu-Glu, Gln-Gln, Asn-Gln, Gln-Asn, Asp-Gln, Gln-Asp, Asn-Glu, Glu-Asn, Asp-Glu, Glu-Asp, Gln-Glu, Glu-Gln, Asp-Asn, Asn-Asp Thr-Thr, Ser-Ser, Thr-Ser, Ser-Thr, Asp-Ser, Ser-Asp, Ser-Asn, Asn-Ser, Gln-Ser, Ser-Gln, Glu-Ser, Ser-Glu, Asp-Thr, Thr-Asp, Thr-Asn, Asn-Thr, Gln-Thr, Thr-Gln, Glu-Thr, Thr-Glu, Cys-Asp, Cys-Lys, Cys-Ser, Cys-Thr, Cys-Orn, Cys-Dab, Cys-Dap, Lys-Lys, Lys-Ser, Lys-Thr, Lys-Orn, Lys-Dab, Lys-Dap, Ser-Lys, Ser-Orn, Ser-Dab, Ser-Dap, Orn-Lys, Orn-Orn, Orn-Ser, Orn-Thr, Orn-Dab, Orn-Dap, Dab-Lys, Dab-Ser, Dab-Thr, Dab-Orn, Dab-Dab, Dab-Dap, Dap-Lys, Dap-Ser, Dap-Thr, Dap-Orn, Dap-Dab, Dap-Dap.

13. The depsipeptide according to claim 1, wherein (X^{AHA})_{b}-(Y), of formula I or (X)_{b1}-(AHA)_{d}-(X)_{b2}-(Y), of formula II has a sequence selected from the group consisting of:
(SEQ ID NO: 38)
LIVA(AHA)K,
(SEQ ID NO: 39)
LIV(AHA)GK,
(SEQ ID NO: 40)
LIVA(AHA)D,
(SEQ ID NO: 41)
LIV(AHA)GD,
(SEQ ID NO: 2)
LIVA(AHA),
(SEQ ID NO: 1)
LIVAG(AHA),
(SEQ ID NO: 42)
ILVA(AHA)K,
(SEQ ID NO: 43)
ILV(AHA)GK,
(SEQ ID NO: 5)
ILVA(AHA),
(SEQ ID NO: 4)
ILVAG(AHA),
(SEQ ID NO: 44)
IVLA(AHA)K,
(SEQ ID NO: 45)
IVL(AHA)GK,
(SEQ ID NO: 46)
AIVA(AHA)S,
(SEQ ID NO: 47)
AIV(AHA)GS,
(SEQ ID NO: 9)
AIVAG(AHA),
(SEQ ID NO: 48)
IV(AHA)D,
IV(AHA),
and
IL(AHA),
wherein AHA refers to α-hydroxy acid.

14. The depsipeptide according to claim 13, wherein (X^{AHA})_{b}-(Y), of formula I or (X)_{b1}-(AHA)_{d}-(X)_{b2}-(Y), of formula II has a sequence selected from the group consisting of:
(SEQ ID NO: 49)
LIVAgK,
(SEQ ID NO: 50)
LIVaGK,
(SEQ ID NO: 51)
ILVAgK,
(SEQ ID NO: 52)
ILVaGK,
(SEQ ID NO: 53)
IVLAgK,
(SEQ ID NO: 54)
IVLaGK,
(SEQ ID NO: 55)
LIVAgD,
(SEQ ID NO: 56)
LIVaGD,
(SEQ ID NO: 57)
AIVAgS
(SEQ ID NO: 58)
AIVaGS
(SEQ ID NO: 59)
IVaD
(SEQ ID NO: 14)
LIVAm,
(SEQ ID NO: 18)
ILVAm,
(SEQ ID NO: 24)
AIVAGm,
IVa,
IVm,
and
ILa,
wherein “g” refers to glycolic acid, “a” refers to lactic acid and “m” refers to malic acid.

15. The depsipeptide according to claim 1, wherein (X)_{b′}-(Y)_{c}-(AHA)_{d} of formula III has a sequence selected from the group consisting of:
(SEQ ID NO: 60)
IVD(AHA),
(SEQ ID NO: 61)
IVK(AHA),
(SEQ ID NO: 62)
ILVAGK(AHA),
(SEQ ID NO: 63)
ILVAGD(AHA),
(SEQ ID NO: 64)
ILVAGS(AHA),
(SEQ ID NO: 65)
LIVAGK(AHA),
(SEQ ID NO: 66)
LIVAGD(AHA),
(SEQ ID NO: 67)
LIVAGS(AHA),
and
(SEQ ID NO: 68)
AIVAGS(AHA),
wherein AHA refers to α-hydroxy acid.

16. The depsipeptide according to claim 15, wherein (X)_{b′}-(Y)_{c}-(AHA)_{d} of formula III has a sequence selected from the group consisting of:
(SEQ ID NO: 69)
IVDa,
(SEQ ID NO: 70)
IVKa,
wherein “a” refers to lactic acid.

17. The depsipeptide according to claim 1, wherein a is 1 and said N-terminal protecting group Z has the general formula —C(O)—R, wherein R is selected from the group consisting of H, unsubstituted or substituted alkyls, and unsubstituted or substituted aryls.

18. The depsipeptide according to claim 1, wherein said N-terminal protecting group Z is an acetyl group.

19. The depsipeptide according to claim 1, wherein said N-terminal protecting group Z is a peptidomimetic molecule, including natural and synthetic amino acid derivatives, wherein the N-terminus of said peptidomimetic molecule is modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

20. The depsipeptide according to claim 1, wherein said C-terminal protecting group Z′ is an amide group or an ester group.

21. (canceled)

22. (canceled)

23. The depsipeptide according to claim 1, wherein said C-terminal protecting group Z′ is a peptidomimetic molecule, including natural and synthetic amino acid derivatives, wherein the C-terminus of said peptidomimetic molecule is modified with a functional group selected from the group consisting of carboxylic acid, amide, alcohol, aldehyde, amine, imine, nitrile, an urea analog, thiol, phosphate, carbonate, sulfate, nitrate, maleimide, vinyl sulfone, azide, alkyne, alkene, carbohydrate, imide, peroxide, ester, thioester, aryl, ketone, sulphite, nitrite, phosphonate and silane.

24.-31. (canceled)

32. A method of preparing a co gel or co-hydrogel, the method comprising:
(a) dissolving at least one depsipeptide as defined in claim 1 in an aqueous solution,
(b) dissolving at least one peptide which has the same sequence as the depsipeptide of step (a), but includes no AHA (“parent peptide”), in an aqueous solution,
(c) mixing the solutions of (a) and (b) and gelating,
(d) obtaining the co-gel or co-hydrogel.

33.-38. (canceled)

39. A co-gel or co-hydrogel comprising:
at least one depsipeptide as defined in claim 1, and at least one parent peptide, i.e. a peptide which has the same sequence as the depsipeptide, but includes no AHA.

40. The co-gel or co-hydrogel of claim 39, wherein the co-gel or co-hydrogel has a lower degradation stability compared to the hydrogel comprising the parent peptide, i.e. the peptide which has the same sequence as the depsipeptide but includes no AHA, but not the depsipeptide.

41. The co gel or co-hydrogel of claim 39, furthermore comprising:
further compound(s), which are encapsulated by the hydrogel or the co-gel or co-hydrogel, wherein said further compound(s) can be selected from:
bioactive molecules or moieties,
such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
label(s), dye(s),
such as imaging contrast agents;
pathogens,
such as viruses, bacteria and parasites;
quantum dots, nano- and microparticles,
or combinations thereof; and/or
cells, which are encapsulated by the hydrogel or the co-gel or co-hydrogel and/or added onto the hydrogel or the co-gel or co-hydrogel after gelation,.
wherein said cells are the same or different, and can be stem cells (adult, progenitor, embryonic and induced pluripotent stem cells), transdifferentiated progenitor cells, and primary cells (isolated from patients) and cell lines (such as epithelial, neuronal, hematopoietic and cancer cells).

42. A pharmaceutical and/or cosmetic composition and/or a biomedical device and/or a surgical implant comprising at least one depsipeptide of claim 1.

43. (canceled)

44. (canceled)

45. A kit of parts, the kit comprising:
a first container with at least one depsipeptide of claim 1, and
a second container with an aqueous solution.

46.-56. (canceled)


SEQUENCE LISTING (71 sequences)

SEQ ID NO: 1 - protein, 6 aa, Artificial Sequence
  (XAHA)b of formula I or (X)b1-(AHA)d-(X)b2 of formula II with Xaa (i.e.AHA) = alpha-hydroxy acid
  misc_feature (6)..(6): Xaa can be any naturally occurring amino acid
LIVAGX

SEQ ID NO: 2 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (5)..(5): Xaa can be any naturally occurring amino acid
LIVAX

SEQ ID NO: 3 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (4)..(4): Xaa can be any naturally occurring amino acid
LIVXG

SEQ ID NO: 4 - protein, 6 aa, Artificial Sequence
  (XAHA)b of formula I or (X)b1-(AHA)d-(X)b2 of formula II with Xaa (i.e.AHA) = alpha-hydroxy acid
  misc_feature (6)..(6): Xaa can be any naturally occurring amino acid
ILVAGX

SEQ ID NO: 5 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (5)..(5): Xaa can be any naturally occurring amino acid
ILVAX

SEQ ID NO: 6 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (5)..(5): Xaa can be any naturally occurring amino acid
IVLAX

SEQ ID NO: 7 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (4)..(4): Xaa can be any naturally occurring amino acid
IVLXG

SEQ ID NO: 8 - protein, 6 aa, Artificial Sequence
  (XAHA)b of formula I or (X)b1-(AHA)d-(X)b2 of formula II with Xaa (i.e.AHA) = alpha-hydroxy acid
  misc_feature (6)..(6): Xaa can be any naturally occurring amino acid
AIVAGX

SEQ ID NO: 9 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (5)..(5): Xaa can be any naturally occurring amino acid
AIVAX

SEQ ID NO: 10 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (4)..(4): Xaa can be any naturally occurring amino acid
AIVXG

SEQ ID NO: 11 - protein, 5 aa, Artificial Sequence
  (XAHA)b of formula I or (X)b1-(AHA)d-(X)b2 of formula II with Xaa (i.e.AHA) = glycolic acid
  misc_feature (5)..(5): Xaa can be any naturally occurring amino acid
LIVAX

SEQ ID NO: 12 - protein, 5 aa, Artificial Sequence
  (XAHA)b of formula I or (X)b1-(AHA)d-(X)b2 of formula II with Xaa (i.e.AHA) = lactic acid
  misc_feature (4)..(4): Xaa can be any naturally occurring amino acid
LIVXG

SEQ ID NO: 13 - protein, 5 aa, Artificial Sequence
  (XAHA)b of formula I or (X)b1-(AHA)d-(X)b2 of formula II with Xaa (i.e.AHA) = malic acid
  misc_feature (5)..(5): Xaa can be any naturally occurring amino acid
LIVAX

SEQ ID NO: 14 - protein, 6 aa, Artificial Sequence
  (XAHA)b of formula I or (X)b1-(AHA)d-(X)b2 of formula II with Xaa (i.e.AHA) = malic acid
  misc_feature (6)..(6): Xaa can be any naturally occurring amino acid
LIVAGX

SEQ ID NO: 15 - protein, 5 aa, Artificial Sequence
  (XAHA)b of formula I or (X)b1-(AHA)d-(X)b2 of formula II with Xaa (i.e.AHA) = glycolic acid
  misc_feature (5)..(5): Xaa can be any naturally occurring amino acid
ILVAX

SEQ ID NO: 16 - protein, 5 aa, Artificial Sequence
  (XAHA)b of formula I or (X)b1-(AHA)d-(X)b2 of formula II with Xaa (i.e.AHA) = lactic acid
  misc_feature (4)..(4): Xaa can be any naturally occurring amino acid
ILVXG

SEQ ID NO: 17 - protein, 5 aa, Artificial Sequence
  (XAHA)b of formula I or (X)b1-(AHA)d-(X)b2 of formula II with Xaa (i.e.AHA) = malic acid
  misc_feature (5)..(5): Xaa can be any naturally occurring amino acid
ILVAX

SEQ ID NO: 18 - protein, 6 aa, Artificial Sequence
  (XAHA)b of formula I or (X)b1-(AHA)d-(X)b2 of formula II with Xaa (i.e.AHA) = malic acid
  misc_feature (6)..(6): Xaa can be any naturally occurring amino acid
ILVAGX

SEQ ID NO: 19 - protein, 5 aa, Artificial Sequence
  (XAHA)b of formula I or (X)b1-(AHA)d-(X)b2 of formula II with Xaa (i.e.AHA) = glycolic acid
  misc_feature (5)..(5): Xaa can be any naturally occurring amino acid
IVLAX

SEQ ID NO: 20 - protein, 5 aa, Artificial Sequence
  (XAHA)b of formula I or (X)b1-(AHA)d-(X)b2 of formula II with Xaa (i.e.AHA) = lactic acid
  misc_feature (4)..(4): Xaa can be any naturally occurring amino acid
IVLXG

SEQ ID NO: 21 - protein, 5 aa, Artificial Sequence
  (XAHA)b of formula I or (X)b1-(AHA)d-(X)b2 of formula II with Xaa (i.e.AHA) = glycolic acid
  misc_feature (5)..(5): Xaa can be any naturally occurring amino acid
AIVAX

SEQ ID NO: 22 - protein, 5 aa, Artificial Sequence
  (XAHA)b of formula I or (X)b1-(AHA)d-(X)b2 of formula II with Xaa (i.e.AHA) = lactic acid
  misc_feature (4)..(4): Xaa can be any naturally occurring amino acid
AIVXG

SEQ ID NO: 23 - protein, 6 aa, Artificial Sequence
  (XAHA)b of formula I or (X)b1-(AHA)d-(X)b2 of formula II with Xaa (i.e.AHA) = malic acid
  misc_feature (6)..(6): Xaa can be any naturally occurring amino acid
AIVAGX

SEQ ID NO: 24 - protein, 5 aa, Artificial Sequence
  (XAHA)b of formula I or (X)b1-(AHA)d-(X)b2 of formula II with Xaa (i.e.AHA) = alpha-hydroxy acid
  misc_feature (4)..(4): Xaa can be any naturally occurring amino acid
ILVXG

SEQ ID NO: 25 - protein, 5 aa, Artificial Sequence
  (X)b′ of formula III
LIVAG

SEQ ID NO: 26 - protein, 5 aa, Artificial Sequence
  (X)b′ of formula III
ILVAG

SEQ ID NO: 27 - protein, 5 aa, Artificial Sequence
  (X)b′ of formula III
LIVAA

SEQ ID NO: 28 - protein, 5 aa, Artificial Sequence
  (X)b′ of formula III
LAVAG

SEQ ID NO: 29 - protein, 5 aa, Artificial Sequence
  (X)b′ of formula III
AIVAG

SEQ ID NO: 30 - protein, 5 aa, Artificial Sequence
  (X)b′ of formula III
GIVAG

SEQ ID NO: 31 - protein, 5 aa, Artificial Sequence
  (X)b′ of formula III
VIVAG

SEQ ID NO: 32 - protein, 5 aa, Artificial Sequence
  (X)b′ of formula III
ALVAG

SEQ ID NO: 33 - protein, 5 aa, Artificial Sequence
  (X)b′ of formula III
GLVAG

SEQ ID NO: 34 - protein, 5 aa, Artificial Sequence
  (X)b′ of formula III
VLVAG

SEQ ID NO: 35 - protein, 4 aa, Artificial Sequence
  (X)b′ of formula III
IVAG

SEQ ID NO: 36 - protein, 4 aa, Artificial Sequence
  (X)b′ of formula III
LIVA

SEQ ID NO: 37 - protein, 4 aa, Artificial Sequence
  (X)b′ of formula III
LIVG

SEQ ID NO: 38 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (5)..(5): Xaa can be any naturally occurring amino acid
LIVAXK

SEQ ID NO: 39 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (4)..(4): Xaa can be any naturally occurring amino acid
LIVXGK

SEQ ID NO: 40 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (4)..(4): Xaa can be any naturally occurring amino acid
LIVXGD

SEQ ID NO: 41 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (4)..(4): Xaa can be any naturally occurring amino acid
LIVXGD

SEQ ID NO: 42 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (5)..(5): Xaa can be any naturally occurring amino acid
ILVAXK

SEQ ID NO: 43 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (4)..(4): Xaa can be any naturally occurring amino acid
ILVXGK

SEQ ID NO: 44 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (5)..(5): Xaa can be any naturally occurring amino acid
IVLAXK

SEQ ID NO: 45 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (4)..(4): Xaa can be any naturally occurring amino acid
IVLXGK

SEQ ID NO: 46 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (5)..(5): Xaa can be any naturally occurring amino acid
AIVAXS

SEQ ID NO: 47 - protein, 6 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (4)..(4): Xaa can be any naturally occurring amino acid
AIVXGS

SEQ ID NO: 48 - protein, 4 aa, Artificial Sequence
  (XAHA)b-(Y)c of formula I or (X)b1-(AHA)d- (X)b2-(Y)c of formula II with Xaa (i.e.AHA) = alpha-hydroxy acid
  misc_feature (3)..(3): Xaa can be any naturally occurring amino acid
IVXD

SEQ ID NO: 49 - protein, 6 aa, Artificial Sequence
  (XAHA)b-(Y)c of formula I or (X)b1-(AHA)d- (X)b2-(Y)c of formula II with Xaa (i.e.AHA) = glycolic acid
  misc_feature (5)..(5): Xaa can be any naturally occurring amino acid
LIVAXK

SEQ ID NO: 50 - protein, 6 aa, Artificial Sequence
  (XAHA)b-(Y)c of formula I or (X)b1-(AHA)d- (X)b2-(Y)c of formula II with Xaa (i.e.AHA) = lactic acid
  misc_feature (4)..(4): Xaa can be any naturally occurring amino acid
LIVXGK

SEQ ID NO: 51 - protein, 6 aa, Artificial Sequence
  (XAHA)b-(Y)c of formula I or (X)b1-(AHA)d- (X)b2-(Y)c of formula II with Xaa (i.e.AHA) = glycolic acid
  misc_feature (5)..(5): Xaa can be any naturally occurring amino acid
ILVAXK

SEQ ID NO: 52 - protein, 6 aa, Artificial Sequence
  (XAHA)b-(Y)c of formula I or (X)b1-(AHA)d- (X)b2-(Y)c of formula II with Xaa (i.e.AHA) = lactic acid
  misc_feature (4)..(4): Xaa can be any naturally occurring amino acid
ILVXGK

SEQ ID NO: 53 - protein, 6 aa, Artificial Sequence
  (XAHA)b-(Y)c of formula I or (X)b1-(AHA)d- (X)b2-(Y)c of formula II with Xaa (i.e.AHA) = glycolic acid
  misc_feature (5)..(5): Xaa can be any naturally occurring amino acid
IVLAXK

SEQ ID NO: 54 - protein, 6 aa, Artificial Sequence
  (XAHA)b-(Y)c of formula I or (X)b1-(AHA)d- (X)b2-(Y)c of formula II with Xaa (i.e.AHA) = lactic acid
  misc_feature (4)..(4): Xaa can be any naturally occurring amino acid
IVLXGK

SEQ ID NO: 55 - protein, 6 aa, Artificial Sequence
  (XAHA)b-(Y)c of formula I or (X)b1-(AHA)d- (X)b2-(Y)c of formula II with Xaa (i.e.AHA) = glycolic acid
  misc_feature (5)..(5): Xaa can be any naturally occurring amino acid
LIVAXD

SEQ ID NO: 56 - protein, 6 aa, Artificial Sequence
  (XAHA)b-(Y)c of formula I or (X)b1-(AHA)d- (X)b2-(Y)c of formula II with Xaa (i.e.AHA) = lactic acid
  misc_feature (4)..(4): Xaa can be any naturally occurring amino acid
LIVXGD

SEQ ID NO: 57 - protein, 6 aa, Artificial Sequence
  (XAHA)b-(Y)c of formula I or (X)b1-(AHA)d- (X)b2-(Y)c of formula II with Xaa (i.e.AHA) = glycolic acid
  misc_feature (5)..(5): Xaa can be any naturally occurring amino acid
AIVAXS

SEQ ID NO: 58 - protein, 6 aa, Artificial Sequence
  (XAHA)b-(Y)c of formula I or (X)b1-(AHA)d- (X)b2-(Y)c of formula II with Xaa (i.e.AHA) = lactic acid
  misc_feature (4)..(4): Xaa can be any naturally occurring amino acid
AIVXGS

SEQ ID NO: 59 - protein, 4 aa, Artificial Sequence
  (XAHA)b-(Y)c of formula I or (X)b1-(AHA)d- (X)b2-(Y)c of formula II with Xaa (i.e.AHA) = lactic acid
  misc_feature (3)..(3): Xaa can be any naturally occurring amino acid
IVXD

SEQ ID NO: 60 - protein, 4 aa, Artificial Sequence
  (X)b′-(Y)c-(AHA)d of formula III with Xaa (i.e.AHA) = alpha-hydroxy acid
  misc_feature (4)..(4): Xaa can be any naturally occurring amino acid
IVDX

SEQ ID NO: 61 - protein, 4 aa, Artificial Sequence
  (X)b′-(Y)c-(AHA)d of formula III with Xaa (i.e.AHA) = alpha-hydroxy acid
  misc_feature (4)..(4): Xaa can be any naturally occurring amino acid
IVKX

SEQ ID NO: 62 - protein, 7 aa, Artificial Sequence
  (X)b′-(Y)c-(AHA)d of formula III with Xaa (i.e.AHA) = alpha-hydroxy acid
  misc_feature (7)..(7): Xaa can be any naturally occurring amino acid
ILVAGKX

SEQ ID NO: 63 - protein, 7 aa, Artificial Sequence
  (X)b′-(Y)c-(AHA)d of formula III with Xaa (i.e.AHA) = alpha-hydroxy acid
  misc_feature (7)..(7): Xaa can be any naturally occurring amino acid
ILVAGDX

SEQ ID NO: 64 - protein, 7 aa, Artificial Sequence
  (X)b′-(Y)c-(AHA)d of formula III with Xaa (i.e.AHA) = alpha-hydroxy acid
  misc_feature (7)..(7): Xaa can be any naturally occurring amino acid
ILVAGSX

SEQ ID NO: 65 - protein, 7 aa, Artificial Sequence
  (X)b′-(Y)c-(AHA)d of formula III with Xaa (i.e.AHA) = alpha-hydroxy acid
  misc_feature (7)..(7): Xaa can be any naturally occurring amino acid
LIVAGKX

SEQ ID NO: 66 - protein, 7 aa, Artificial Sequence
  (X)b′-(Y)c-(AHA)d of formula III with Xaa (i.e.AHA) = alpha-hydroxy acid
  misc_feature (7)..(7): Xaa can be any naturally occurring amino acid
LIVAGDX

SEQ ID NO: 67 - protein, 7 aa, Artificial Sequence
  (X)b′-(Y)c-(AHA)d of formula III with Xaa (i.e.AHA) = alpha-hydroxy acid
  misc_feature (7)..(7): Xaa can be any naturally occurring amino acid
LIVAGSX

SEQ ID NO: 68 - protein, 7 aa, Artificial Sequence
  (X)b′-(Y)c-(AHA)d of formula III with Xaa (i.e.AHA) = alpha-hydroxy acid
  misc_feature (7)..(7): Xaa can be any naturally occurring amino acid
AIVAGSX

SEQ ID NO: 69 - protein, 4 aa, Artificial Sequence
  (X)b′-(Y)c-(AHA)d of formula III with Xaa (i.e.AHA) = lactic acid
  misc_feature (4)..(4): Xaa can be any naturally occurring amino acid
IVDX

SEQ ID NO: 70 - protein, 4 aa, Artificial Sequence
  (X)b′-(Y)c-(AHA)d of formula III with Xaa (i.e.AHA) = lactic acid
  misc_feature (4)..(4): Xaa can be any naturally occurring amino acid
IVKX

SEQ ID NO: 71 - protein, 6 aa, Artificial Sequence
  parent peptide
ILVAGK